(19) United States
(12) Patent Application Publication
(10) Pub. No.: US 2004/0193450 A1
(43) Pub. Date: Sep. 30, 2004

(54) Healthcare record classification system

(76) Inventor: Robert Ernest Knapp, Berwyn, PA (US)

Correspondence Address: Alexander J. Burke, Intellectual Property Department, 5th Floor, 170 Wood Avenue South, Iselin, NJ, 08830, US

(21) Appl. No.: 10/681,709

(22) Filed: Oct. 8, 2003

Related U.S. Application Data
(60) Provisional application No. 60/457,055, filed on Mar. 24, 2003.

(51) Int. Cl. G06F 17/60; A61B 5/00

(52) U.S. Cl. 705/2; 705/3; 600/300

(57) ABSTRACT

Certain exemplary embodiments comprise a method for associating a diagnostic code to a record of a patient visit, the method comprising receiving a visit record comprising a first diagnostic code derived by using a first assignment system, retrieving rules for processing the visit record to determine a second diagnostic code compatible with a second assignment system, processing the visit record and the first diagnostic code using rules to provide the visit record comprising the second diagnostic code, and initiating communication of the visit record including the second diagnostic code compatible with the second assignment system to a destination system.

### CROSS-REFERENCE TO RELATED APPLICATIONS

This application claims priority to pending U.S. Provisional Patent Application Serial No. 60/457,055 (Applicant Docket No. 03P04397US), filed 24 Mar. 2003.

### BACKGROUND

The development and management of information relating to the provision of health care services is important to assure quality of care, proper reimbursement, and/or managerial effectiveness. When a patient visits a health care provider or receives a health-related service, a record of that visit is created, the record including a coded set of characteristics that describe the visit. For example, a visit record comprises coded representations of an identity of the patient and/or the date of service. The visit record further comprises a diagnostic code related to the type of service provided. The diagnostic code is generated by a diagnostic code assignment system. Numerous diagnostic code assignment systems are known.

Diagnostic codes are grouped for analytical or managerial purposes, via, for example, grouping software, which provides a means of automatically grouping visit records for comparable diagnostic codes or types of services. For example, visit records for cold and flu diagnoses are grouped together. Diagnostic codes and grouping rules change over time. Known systems do not convert records having previously assigned diagnostic codes and do not re-group values when updated code assignment rules are released.

A system according to the principles of the invention addresses the identified deficiencies and associated problems.

### SUMMARY

Certain exemplary embodiments comprise a method for associating a diagnostic code to a record of a patient visit, the method comprising receiving a visit record comprising a first diagnostic code derived by using a first assignment system, retrieving rules for processing the visit record to determine a second diagnostic code compatible with a second assignment system, processing the visit record and the first diagnostic code using rules to provide the visit record comprising the second diagnostic code, and initiating communication of the visit record including the second diagnostic code compatible with the second assignment system to a destination system.

### BRIEF DESCRIPTION OF THE DRAWINGS

The claims and their wide variety of potential embodiments will be more readily understood through the following detailed description, with reference to the accompanying drawings in which:

FIG. 1 is a flow diagram of an exemplary embodiment of a method 1000 for processing a visit record;

FIG. 2 is a block diagram of an exemplary embodiment of a rendering of a user interface 2000;

FIG. 3 is a block diagram of an exemplary embodiment of a system 3000 usable for processing a record from a visit of a patient;

FIG. 4 is a block diagram of an exemplary embodiment of a method of use 4000 for processing a record from a visit of a patient;

FIG. 5 is a flow diagram of an exemplary embodiment of a method of use 5000 for processing a record from a visit of a patient;

FIG. 6 is an exemplary embodiment of a flow diagram of a method of use 6000 for processing a record from a visit of a patient;

FIG. 7 is a block diagram of an exemplary embodiment of a system 7000;

FIG. 8 is a block diagram of an exemplary embodiment of an information device 8000;

FIG. 9 is a block diagram of an exemplary embodiment of a machine readable medium 9000; and

FIG. 10 is a block diagram of an exemplary embodiment of a machine readable medium 10000.

### DETAILED DESCRIPTION

As used herein in the context of a patient visit, the term “characteristics” means any feature or element defining or describing the patient visit. Characteristics comprise a principal diagnosis, a principal procedure, an age at admission, a gender, a presence of particular secondary diagnoses, a delivery of certain chargeable services, and/or a length of patient stay, etc.

As used herein, the term “computer program” means a sequence of instructions that an information device interprets and executes.

As used herein, the term “consistent” as applied to a patient visit record diagnostic code means that patient visits comprising the same diagnosis and treatment receive the same diagnostic code, regardless of when the patient visit occurred.

As used herein, the term “controller” means a device for processing machine-readable instruction. A controller might be a central processing unit, a local controller, a remote controller, parallel controllers, and/or distributed controllers, etc. The controller might be a general-purpose microcontroller, such the Pentium III series of microcontrollers manufactured by the Intel Corporation of Santa Clara, Calif. In another embodiment, the controller might be an Application Specific Integrated Circuit (ASIC) or a Field Programmable Gate Array (FPGA) that has been designed to implement in its hardware and/or software at least a part of an embodiment disclosed herein.

As used herein, the term “database” means one or more structured sets of persistent data, usually associated with software to update and query the data. A simple database might be a single file containing many records, each of which is structured using the same set of fields.

As used herein, the term “data table” means a set of data arranged in rows and columns.

As used herein, the term “diagnostic code” means at least one character corresponding to a reason for a patient visit and/or treatment. The diagnostic code is assignable based upon a reimbursement requirement by a third party. Alternatively, the diagnostic code is assignable based upon a need to analyze the utilization of medical resources.

As used herein the terms “diagnostic code assignment system”, “Diagnostic code sets”, or “standard diagnostic code set” means a plurality of sets of characters and/or symbols organized to correspond to a plurality of maladies and/or treatments of a patient. A diagnostic code assignment system comprises for example, ICD (International Classification of Diseases) codes, 9th Edition, Clinical Modification, (ICD-9-CM), Volumes 1, 2 and 3, as well as ICD-10 maintained and distributed by the U.S. Health and Human Services department. The code sets also include code sets compatible with HCPCS (Health Care Financing Administration Common Procedure Coding System), NDC (National Drug Codes), CPT-4 (Current Procedural Terminology), and Fourth Edition CDPN (Code on Dental Procedures and Nomenclature). Further the code sets and terms include code sets compatible with SNOMED-RT “Systematicized Nomenclature of Medicine, Reference Terminology” by the College of American Pathologists, UMLS (Unified Medical Language System), by the National Library of Medicine, LOINC Logical Observation Identifiers, Names, and Codes Regenstrief Institute and the Logical Observation Identifiers Names and Codes (LOINC®) Committee, Clinical Terms also known as “Read Codes”, DIN Drug Identification Numbers, Reimbursement Classifications including DRGs (Diagnosis Related Groups). The code sets also include code sets compatible with CDT Current Dental Terminology, NIC (Nursing intervention codes) and Commercial Vocabulary Services (such as HealthLanguage by HealthLanguage Inc., by Apelon Inc.) and other code sets used in healthcare.

As used herein, the term “grouper”, or “standard grouper” means a set of machine-readable instructions for clustering patient visit records based upon similar characteristics. Groupers comprise a CMS Grouper, a Champus Grouper, an All-Patient DRG Grouper and/or a United States state associated Grouper, etc.

As used herein, the term “information device” means any device capable of processing information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as a Handspring Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware selectronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device comprises well-known components such as one or more interfaces to a network, one or more processors, one or more memories containing instructions, and/or one or more input/output (I/O) devices, etc.

As used herein, the term “processor” means any device and/or set of machine-readable instructions for performing a specific task. A processor is any one or combination of hardware, and/or software. A processor acts upon information by manipulating, analyzing, modifying, converting, transmitting the information to an information device, and/or routing the information to a device enabling input and or output. A processor resides on and uses the capabilities of a controller.

As used herein, the term “relational database” means a database having data that is contained in either of at least two related files.

As used herein, the term “rendered” means made perceptible to a human, for example as data, commands, text, graphics, audio, video, animation, and/or hyperlinks, etc., such as via any visual and/or audio means, such as via a display, a monitor, selectric paper, an ocular implant, a speaker, a cochlear implant, etc.

As used herein, the term “user interface” means any device accessible to the user comprising at least one user interface elements. As used herein, the term “user interface elements” means at least one of a plurality of fields rendering information and/or requesting information from the user. User interface elements comprise any known user interface structure, including for example, a window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, image, icon, button, control, dial, slider, scroll bar, cursor, status bar, stepper, and/or progress indicator etc.

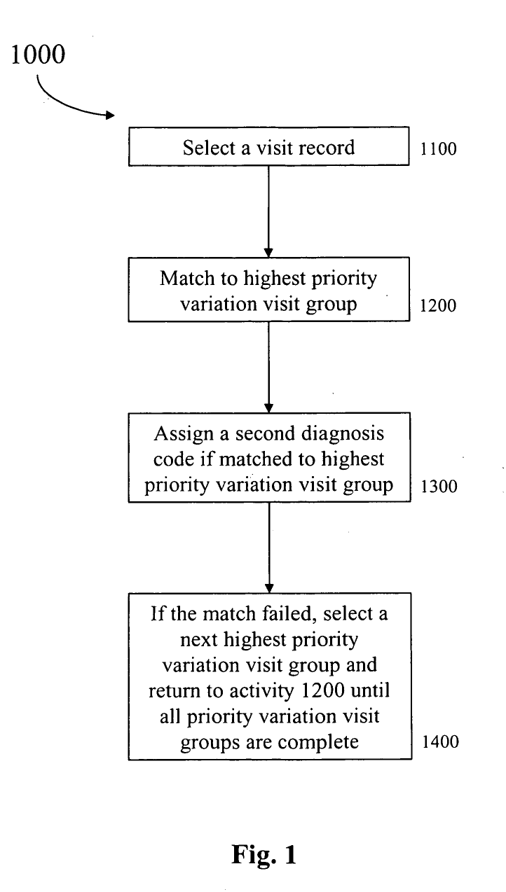

FIG. 1 is a flow diagram of an exemplary embodiment of a method 1000 for processing a visit record. The visit record is relatable to a patient visit to a health care provider. The visit record comprises a set of characteristics. At activity 1100, the visit record, comprising a diagnostic code, is selected from a plurality of visit records. In certain exemplary embodiments, the plurality of visit records comprise records from a single medical provider or office. Alternatively, the plurality of visit records comprises records from a large hospital and/or a plurality of medical providers.

At activity 1200, the visit record, comprising a first diagnostic code, is compared to a highest priority variation visit group. In certain exemplary operative embodiments, the highest priority variation visit group comprises a set of diagnostic codes that are correlatable to treatments and/or diagnoses that are closely enough related to be grouped for analytical and/or reporting purposes. The highest priority variation visit group is characterizable by the severity of patient maladies comprised by patients therein. For example, a terminal cancer patient is assignable to the highest priority variation visit group. Similarly, a patient with a cold is assignable to a lower priority variation visit group. For each customizer grouper scheme in the list, the processor attempts to match the visit record with each variation visit cluster, starting with the highest priority, until a match is found. Comparing the first diagnostic code to the highest priority variation visit group systematically provides a list of visit records processed and clustered according to their priority, the list of visit records is usable for further reporting and analysis.

At activity 1300 the visit record is assigned a second diagnostic code if the record is matched to highest priority variation visit group. The second diagnostic code is usable to cluster a set of visit records in a consistent manner across time. When a match is found, the processor assigns the diagnostic code defined for the variation visit cluster for the customizer grouper scheme to the visit record. Alternatively, when a match is found, the processor replaces the first diagnostic code with the second diagnostic code. If no match is found, the first diagnostic code remains as assigned by the source grouper scheme. Activity 1300 assures that at least one diagnosis code is assigned to a patient visit for informational reporting and analysis.

At activity 1400, a next highest priority variation visit group is selected and the activities beginning at activity 1200 are reexecuted until a match has been found. In certain exemplary embodiments, sequentially examining the visit record across all variation visit groups categorizes the visit record into a cluster. Alternatively, in certain exemplary embodiments, the visit record passes through all variation visit groups without categorization into a cluster.

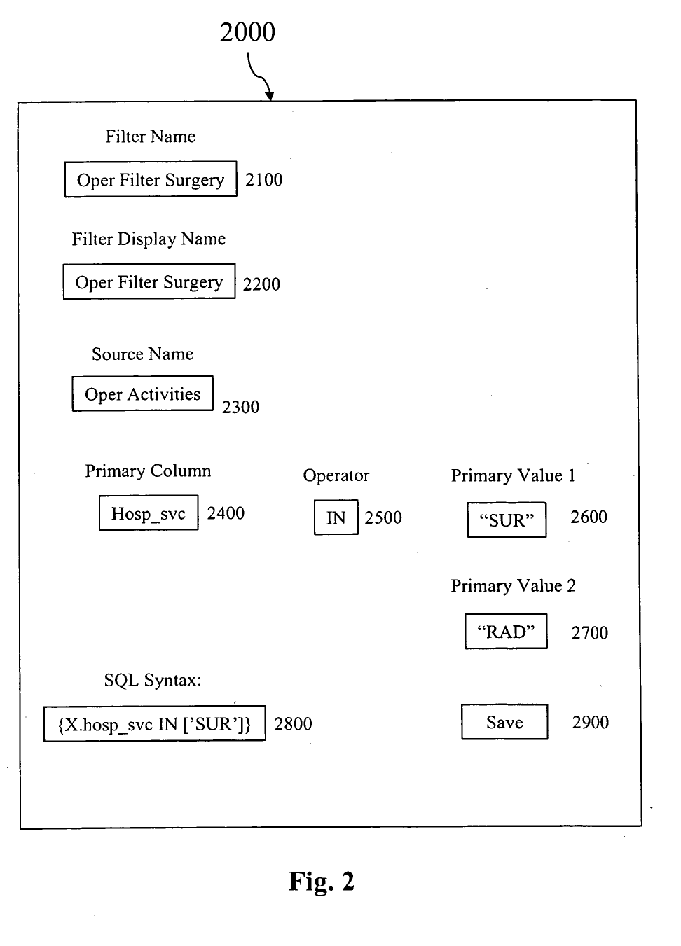

FIG. 2 is a block diagram of an exemplary embodiment of a rendering of a user interface 2000. User interface 2000 is selectable using a GUI interface based upon a Windows operating system standard. Alternatively, user interface 2000 is rendered using an interface standard based upon a Linux, Unix, and/or an Apple Mac OS, etc. system standard. User interface 2000 comprises a plurality of user interface elements. In certain exemplary embodiments, information supplied via user interface 2000 provides information that facilitates a process to relate a second diagnostic code to a visit record comprising a first diagnostic code.

User interface element 2100 accepts a user's input corresponding to a user selection of a filter. The filter is a software rule corresponding to a grouping category usable for clustering the visit record with other visit records sharing a common characteristic. The filter is usable to restrict and/or assist in the determination of the second diagnostic code corresponding to the visit record.

User interface element 2200 accepts a user's input corresponding to a user selection of a filter display name. In certain exemplary embodiments, the filter display name is the same as the filter name at first user interface element 2100. Alternatively, the filter display name is selected to be different from the filter name at first user interface element 2100. The filter display name allows a user selection of a more descriptive display name than the filter name at first user interface element 2100.

User interface element 2300 accepts a user's input corresponding to a user selection of a source name. The source name is indicative of a location of visit records. Alternatively, the source name is indicative of a location a rule. The selection of a source name defines and/or restricts a set of rules selectable to relate the second diagnostic code to the visit record.

User interface element 2400 accepts a user's input corresponding to a user selection of a primary column. The primary column name is indicative of a characteristic of the visit record. The primary column name is indicative of a building for performing medical services. For example, the primary column name is a hospital, a clinic, a medical center, a doctor's office, and/or a patient's home, etc. The user selection of the primary column provides information that further defines the second diagnostic code relatable to the visit record.

User interface element 2500 accepts a user's input corresponding to a user selection of an operator. The operator is indicative of a relationship between the Primary Column and the Primary Value 1 and in certain operative embodiments, the Primary Value 2. For example, the operator comprises values such as =, <, IN, or BETWEEN. When taken together with the Primary Column, Primary Value 1 and, optionally, Primary Value 2 the operator further refines a search for the second diagnostic code that is relatable to the visit record.

User interface element 2600 accepts a user's input corresponding to a user selection of a primary value 1. Primary value 1 is indicative of a characteristic of the visit record. Primary value 1 is indicative of a broad category of medical services performable during the visit. For example, primary value 1 indicates surgery, diagnostic testing, a brief examination, radiation treatments, and/or chemotherapy, etc. Primary value 1 further defines the second diagnostic code that is relatable to the visit record.

User interface element 2700 accepts a user's input corresponding to a user selection of a primary value 2. Primary value 2 is indicative of a characteristic of the visit record. Primary value 2 is indicative of a broad category of medical services performable during the visit. For example, primary value 2 indicates surgery, diagnostic testing, a brief examination, radiation treatments, and/or chemotherapy, etc. Primary value 2 further defines a second diagnostic code that is relatable to the visit record. Primary Value 2 is desirable, for example, when the patient requires more than one category of medical services. User interface element 2800 is informative to a user regarding the nature and scope of an inquiry to a database and/or data table associable with the relation of the second diagnostic code to the visit record.

User interface element 2800 displays a command usable to search and process a plurality of visit records. User interface element 2800 is displayed in a form reflecting a structured query language (SQL) query, an object query language (OQL), a segment of program code written using a version of Basic as a programming language, and/or any a segment of program code in the C+ programming language, etc.

User interface element 2900 allows a user to save the inputs to the filter.

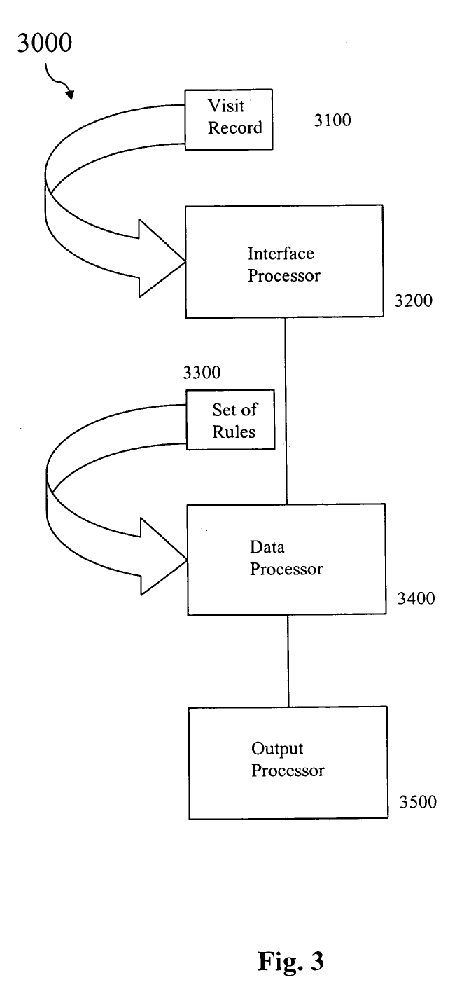

FIG. 3 is a block diagram of an exemplary embodiment of a system 3000 usable for processing a record from a visit of a patient. In certain exemplary embodiments, a system 3000 comprises an interface processor 3200. Interface processor 3200 receives a visit record 3100. Visit record 3100 is a summary of information relatable to a patient visit to a medical provider. Visit record 3100 comprises a plurality of characteristics. Visit record 3100 comprises a first diagnostic code corresponding to a first assignment system. Visit record 3100 is one of a plurality of visit records.

Interface processor 3200 receives visit record 3100 from a program such as a patient accounting system, a patient records system, and/or any customized software for transmitting visit record 3100 to interface processor 3200, etc. Alternatively, interface processor 3200 obtains visit record 3100 from a storage location, or the user provides visit record 3100 to interface processor 3200. In certain exemplary embodiments, interface processor 3200 provides the visit record to an information device for further processing. In an alternative embodiment, interface processor 3200 provides the visit record to another processor for further analysis and/or modification.

A set of rules 3300 is provided to a data processor 3400. Set of rules 3300 is storable in a table, a database, a relational database, and/or a computer program, etc. Interface processor 3200 provides visit record 3100 to data processor 3400. Data processor 3400 processes visit record 3100 applying set of rules 3300. Set of rules 3300 comprises a second assignment system comprising diagnostic codes at least some of which are different than those corresponding to the first assignment system. In certain operative embodiments, a diagnostic code assignable from the first system corresponds to a diagnostic code assignable from the second assignment system. In other operative embodiments, a diagnostic code assignable from the first system does not correspond to a diagnostic code assignable from the second assignment system. In certain operative embodiments, a second assignment system assigns a second diagnostic code to a visit record. Set of rules 3300 is applicable to provide a second diagnostic code, corresponding to a second assignment system, relatable to the visit record.

Data processor 3400 processes visit record 3100 by providing a second diagnostic code responsive to the second assignment system. Data processor 3400 is a processor; usable in certain exemplary operative embodiments to modify visit record 3100. Data processor 3400 is usable to provide the second diagnostic code corresponding to the visit record.

Output processor 3500 further processes visit record 3100 comprising the second diagnostic code to provide information in a format suitable for the user. Output processor 3500 is usable in certain exemplary operative embodiments to provide a representation of visit record 3100 to the user. Output processor 3500 provides information to a device enabling input and or output.

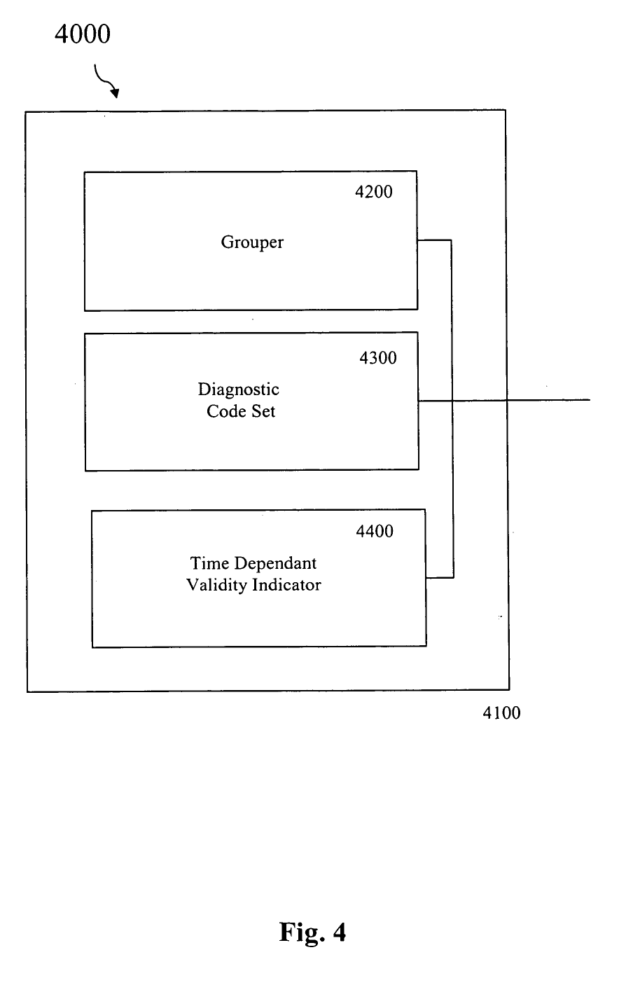

FIG. 4 is a block diagram of an exemplary embodiment of a method of use 4000 for processing a record from a visit of a patient. Certain exemplary embodiments comprise a machine readable medium 4100. Machine readable medium 4100 stores grouper 4200, a diagnostic code set 4300, and/or a time dependent validity indicator 4400 relatable to diagnostic code set 4300.

Diagnostic code set 4300 is comprised of at least one of a standard list of diagnostic code assignment systems. A diagnostic code set provides code information suitable for providing the second diagnostic code corresponding to the visit record.

Time dependent validity indicator 4400 comprises a start date and an end date for which diagnostic code set 4300 is valid. Time dependent validity indicator 4400 provides information about whether diagnostic code set 4300 is valid for a particular time period. Time dependent validity indicator 4400 determines a current validity of a first diagnostic code corresponding to the visit record.

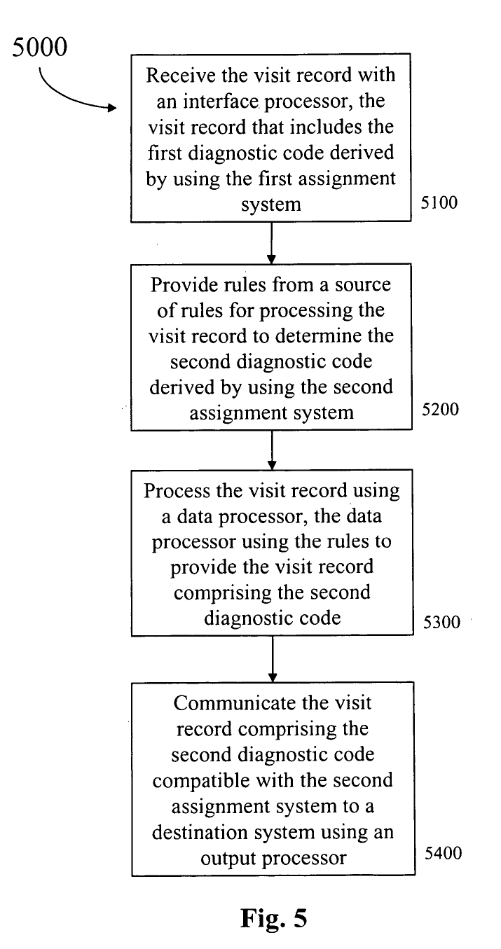

FIG. 5 is a flow diagram of an exemplary embodiment of a method of use 5000 for processing a record from a visit of a patient. At activity 5100, an interface processor receives a visit record that includes a first diagnostic code derived by using a first assignment system. In certain operative embodiments the first diagnostic code is a null code. The first assignment system is any diagnostic code assignment system. The first assignment system is compatible with at least one grouper. Receiving the visit record provides information to allow additional processing in providing the second diagnostic code assignable to the visit record. In certain operative embodiments the second diagnostic code is assignable wherein the first diagnostic code is a null diagnostic code. Further, in certain operative embodiments, the second assignment system replaces the null diagnostic code with a non-null diagnostic code. In certain other exemplary embodiments a visit record is assignable to a group or a cluster responsive to the first diagnostic code and/or the second diagnostic code.

At activity 5200, a source of rules provides rules for processing the visit record to determine a second diagnostic code derived by using a second assignment system. The rules are processable to provide the second diagnostic code using the second assignment system corresponding to the visit record. The rules comprise time dependent rule sets for processing the visit record to determine the second diagnostic code derived by using the second assignment system. The time dependent rule sets determine the validity of the first diagnostic code and/or provide the second diagnostic code corresponding to the visit record

At activity 5300, a data processor processes the visit record using the rules to provide a visit record incorporating the second diagnostic code. The rules are storable in a relational database, a database, a spreadsheet, computer programming code, and/or a data table, etc. The processing is responsive to a user selection of a diagnostic code set and/or a user selection of a grouper. In certain exemplary operative embodiments, the data processor provides the second diagnostic code corresponding to the visit record.

At activity 5400, a destination system, such as an output processor, communicates the visit record comprising the second diagnostic code compatible with the second assignment system to a device enabling input and or output or an information device. Communicating the visit record comprising the second diagnostic code facilitates clustering the visit record into groups and/or rendering information to the user.

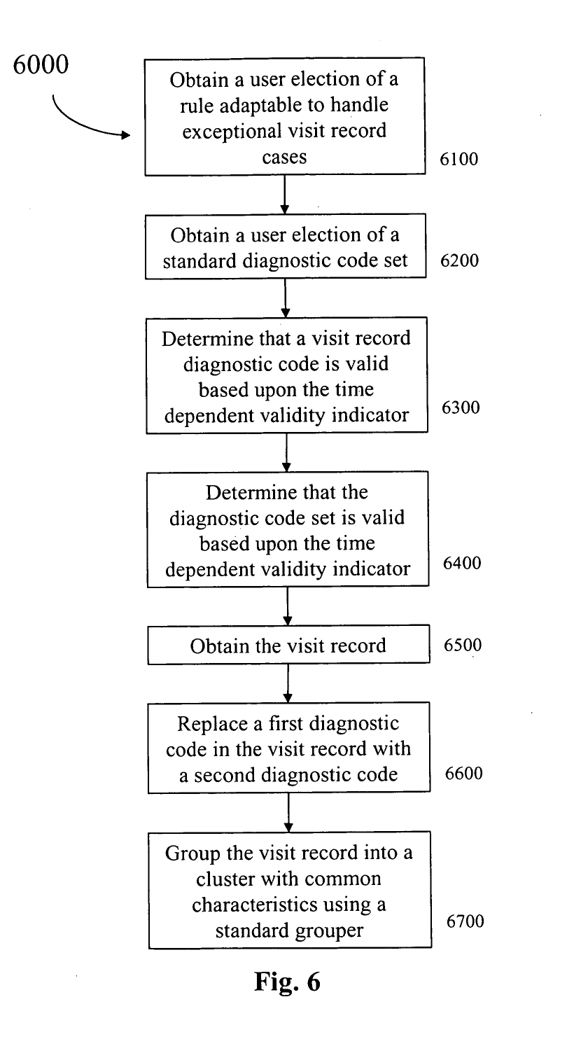

FIG. 6 is a flow diagram of an exemplary embodiment of a method of use 6000 for processing a record from a visit of a patient. At activity 6100, an exemplary embodiment obtains a user selection corresponding to a rule to handle exceptional cases of patient visits. An exceptional case occurs when the first diagnostic code cannot be provided due to an unusual patient malady and/or treatment. Alternatively, the exceptional case occurs when the visit record contains an error. An error can result from improper data transcription, a flaw in the first assignment system, a malfunction in the storage of the visit record, and/or improper translation of the visit record, etc. The second diagnostic code corresponding to the visit record in an exceptional case allows the exceptional case to be clustered and/or grouped.

At activity 6200, an exemplary embodiment receives data identifying a user selection of a diagnostic code set. The diagnostic code set is selectable from a plurality of diagnostic code assignment systems. The diagnostic code assignment systems comprise at least one standard diagnostic code assignment system. The diagnostic code set provides the second diagnostic code corresponding to the visit record.

At activity 6300, an exemplary embodiment determines that the visit record is valid based upon a first time dependent validity indicator. The first time dependent validity indicator is indicative of the validity of the first diagnostic code and/or the second diagnostic code corresponding to the visit record. The first time dependent validity indicator comprises a start date and an end date relating to the validity of the first diagnostic code, the first assignment system, the second diagnostic code, and/or the second assignment system. The first time dependent validity indicator assures that a set of diagnostic codes correlated to a set of visit records are consistent with each other.

At activity 6400, an exemplary embodiment determines that the diagnostic code set is valid based upon a second time dependent validity indicator. The second time dependent validity indicator comprises a start date and an end date. The validity of the diagnostic code set renders the diagnostic code set provides the second diagnostic code corresponding to the visit record. The second time dependent validity indicator determines if the data of a patient visit falls between the time validity indicator start date and the time validity indicator end date. The second time validity indicator assures that a diagnostic code, valid for the date of the visit record, is provided corresponding to the visit record.

At activity 6500, an exemplary embodiment receives data representing a visit record. The data representing a visit record is obtainable, in certain operative embodiments by: reading a data storage medium, transferring from an information device, transferring by a processor, or accepting a user input. In certain exemplary operative embodiments, obtaining the visit record provides information for the determination of the second diagnostic code.

At activity 6600, an exemplary embodiment replaces the first diagnostic code in the visit record with the second diagnostic code responsive to the grouper selected by the user. Alternatively, the second diagnostic code is provided corresponding to the visit record without replacing the first diagnostic code. The second diagnostic code allows visit records to be clustered by the grouper.

At activity 6700, the visit record is grouped or clustered with other visit records comprising common characteristics. The visit records are grouped using a standard grouper. The visit records is grouped or clustered for billing purposes.

Alternatively, the visit records is grouped or clustered in order to improve organizational, and/or resource allocation, effectiveness.

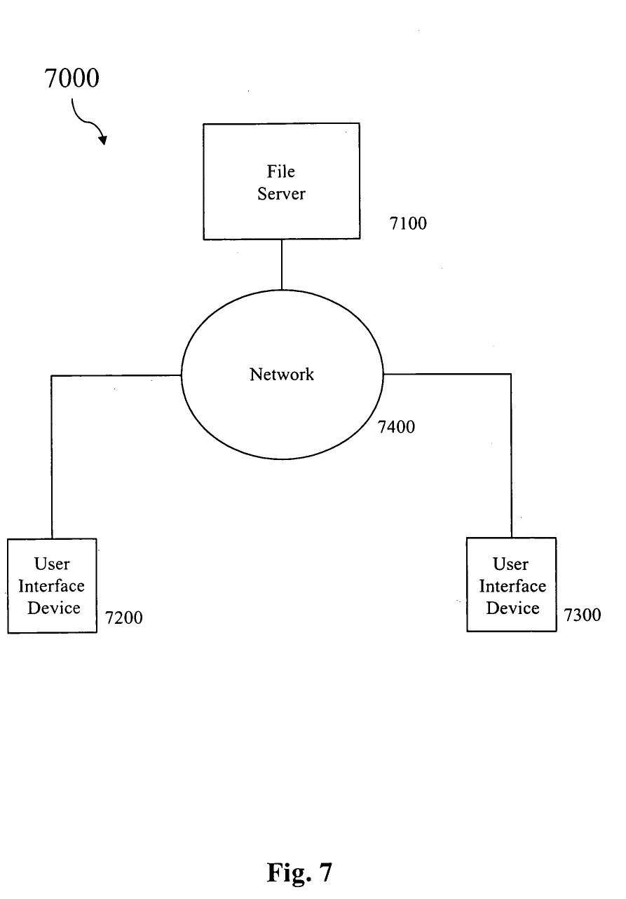

FIG. 7 is a block diagram of an exemplary embodiment of a system 7000. As illustrated, system 7000 comprises at least one file server 7100, which is an information device. File server 7100 provides continuous processing, batch processing, and/or storage of large quantities of information. File server 7100 acts as a server in a client-server relationship with user interface device 7200, 7300. Visit records are storable and categorizable on file server 7100.

User interface device 7200, 7300, which is an information device, allows users to communicate and/or interact with file server 7100 and/or other user interface devices. As used herein “interact” means receiving alerts or notifications, providing user input, reviewing data, revising or switching programs, examining processing algorithms, and/or modifying graphics displays, etc. User interface device 7200, 7300 allow a user to enter, analyze, process, and/or maintain visit records.

In certain exemplary embodiments, file server 7100 is coupled to a user interface device 7200, 7300 via a network 7400. Network 7400 is a public, private, circuit-switched, packet-switched, virtual, radio, telephone, cellular, cable, DSL, satellite, microwave, AC power, twisted pair, ethernet, token ring, LAN, WAN, Internet, intranet, wireless, Wi-Fi, BlueTooth, Airport, 802.11a, 802.11b, 802.11g, and/or any equivalents thereof, etc., network. Network 7400 further comprises one or more interfaces to a network. A device interfacing to a network comprises a telephone, a cellular phone, a modem, a cellular modem, a telephone data modem, a fax modem, a wireless transceiver, an ethernet card, a cable modem, a digital subscriber line interface, a bridge, a hub, a router, or other similar device. A device interfacing to a network allows a user, via a remote user interface device, to communicate with file server 7100, and/or user interface device 7200, 7300.

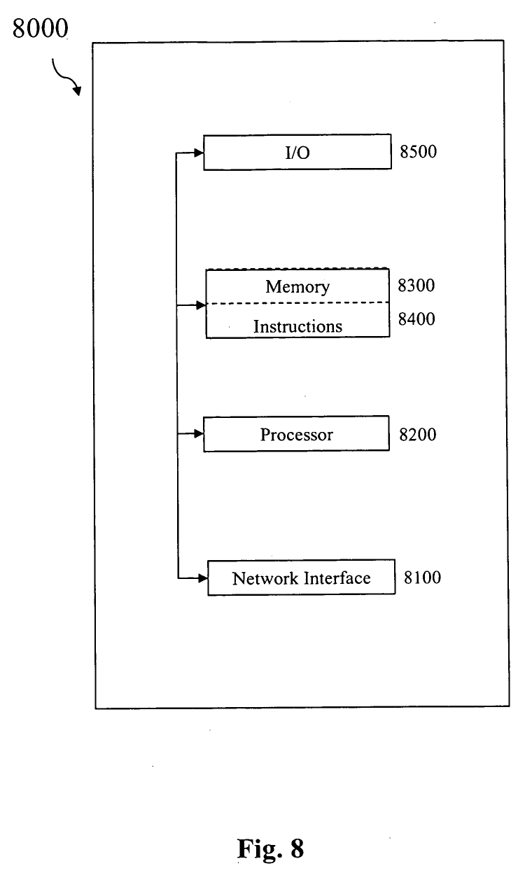

FIG. 8 is a block diagram of an exemplary embodiment of an information device 8000. Information device 8000 includes well-known components such as one or more devices interfacing to a network 8100, one or more processors 8200, one or more memories 8300 containing instructions 8400 and/or data, and/or one or more input/output (I/O) devices 8500, etc. In certain operative embodiments information device 8000 is an interface processor 3200, a data processor 3400, and/or an output processor 3500 as in FIG. 3.

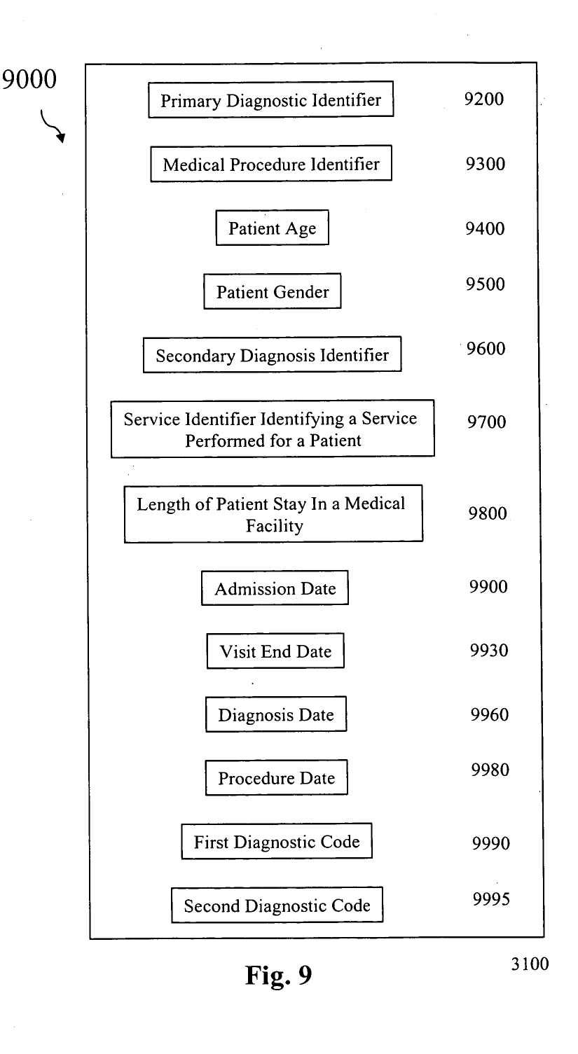

FIG. 9 is a block diagram of an exemplary embodiment of a machine readable medium 9000. Machine readable medium 9000 comprises a visit record 3100. Visit record 3100 comprises a primary diagnosis identifier 9200, a medical procedure identifier 9300, a patient age 9400, a patient gender 9500, a secondary diagnosis identifier 9600, a service identifier identifying a service performed for a patient 9700, a length of patient stay 9800, an admission date 9900, a visit end date 9930, a diagnosis date 9960, and a procedure date 9980. A health care provider typically provides information comprised in visit record 3100 responsive to a patient visit.

Machine readable medium 9000 further comprises a first diagnostic code 9990 and a second diagnostic code 9995. First diagnostic code 9990 is assignable responsive to a health care provider's findings during a patient visit. In certain exemplary embodiments a health care provider assigns first diagnostic code 9990 using a first assignment system. In certain operative embodiments first diagnostic code 9990 is automatically assigned responsive to information otherwise comprised in visit record 3100. Second diagnostic code 9995 is assignable using a second assignment system responsive to the health care provider's findings during a patient visit. In certain exemplary embodiments, second diagnostic code 9995 is assigned automatically responsive to information otherwise comprised in visit record 3100.

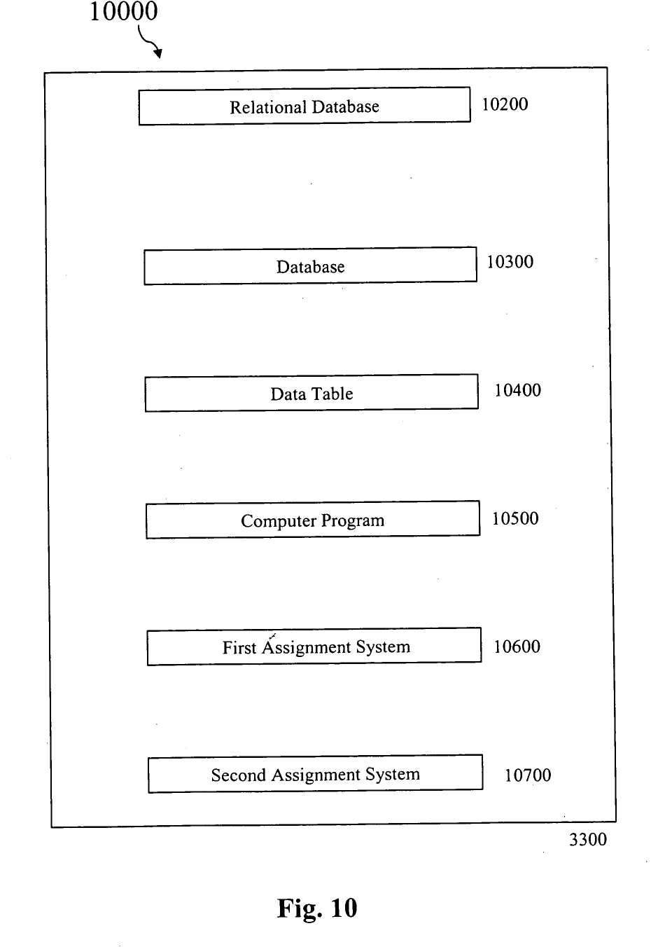

FIG. 10 is a block diagram of an exemplary embodiment of a machine readable medium 10000. Machine readable medium 10000 comprises a set of rules 3300. In certain operative embodiments set of rules 3300 comprises diagnostic code set 4300 from FIG. 4. Set of rules 3300 may be implemented within a relational database 10200. Relational database 10200 comprises a plurality of visit records. The plurality of visit records, for example, comports with information comprised as in visit record 9000 from FIG. 9. Set of rules 3300 further comprise a database 10400, database 10400 also comprises a plurality of visit records. Set of rules 3300 further comprises data table 10400. Data table 10400 also comprises a plurality of visit records. Set of rules 3300 further comprises a computer program 10500 in certain exemplary embodiments. Computer program 10500 comprises a plurality of processors such as, for example, interface processor 3200 from FIG. 3. Set of rules 3300 further comprises a first assignment system 10600 and/or a second assignment system 10700. Assignment system 10600, 10700 is any diagnostic code assignment system.

Still other embodiments will become readily apparent to those skilled in this art from reading the above-recited detailed description and drawings of certain exemplary embodiments.

What is claimed is:

1. A system for associating a diagnostic code to a visit record of a patient visit, comprising:
an interface processor for receiving a visit record comprising a first diagnostic code derived by using a first assignment system;
a source of rules for processing said visit record to determine a second diagnostic code compatible with a second assignment system;
a data processor for processing said visit record and said first diagnostic code using said rules to provide said visit record including said second diagnostic code; and
an output processor for processing said visit record including said second diagnostic code compatible with said second assignment system to be suitable for output to a user.

2. A system according to claim 1, wherein said data processor processes said rules to determine said second diagnostic code compatible with said second assignment system using a plurality of information elements in said visit record including at least one of, (a) a primary diagnosis identifier, (b) a medical procedure identifier, (c) a patient age, (d) a patient gender, (e) a secondary diagnosis identifier, (f) a service identifier identifying a service performed for a patient, (g) a length of patient stay in a medical facility, (h) an admission date, (i) a visit end date, (j) a diagnosis date and (k) a procedure date.

3. A system according to claim 1, wherein said first diagnostic code equals said second diagnostic code.

4. A system according to claim 1, wherein said rules include sets of rules associated with particular time periods of validity for processing said visit record to determine said second diagnostic code compatible with said second assignment system valid during a particular time period, and an individual set of rules has a time period of validity determined by a start date and an end date, and said data processor processes said visit record and said first diagnostic code using said rules to provide said visit record including said second diagnostic code valid for a particular time period encompassing a date of said visit.

5. A system according to claim 1, wherein said interface processor receives said visit record wherein said first diagnostic code is a null code, and said data processor processes said visit record, said rules to provide said visit record including said second diagnostic code.

6. A system according to claim 1, wherein said second assignment system comprises a predetermined system of rules for assigning said second diagnostic code to said visit record based on characteristics of said visit as determined from information contained in said visit record.

7. A system according to claim 6, wherein said second assignment system comprises at least one of, (a) a CMS Grouper, (b) a Champus Grouper, (c) an All-Patient DRG Grouper and (d) a United States state associated Grouper.

8. A system according to claim 1, wherein said second diagnostic code is derived from a code set including at least one of: (a) ICD-9-CM, (b) ICD-10, (c) HCPCS, (d) NDC, (e) CPT-4, (f) CDPN, (g) SNOMED-RT, (h) UMLS, (i) LOINC (j) “Read Codes”, (k) DIN, (l) CDT, (m) NIC, and (n) DRGs Diagnosis Related Groups.

9. A system according to claim 1, wherein said data processor uses said rules, for, identifying whether said first diagnostic code is incompatible with said second assignment system and if said first diagnostic code is incompatible, assigning said second diagnostic code to be compatible with said second assignment system.

10. A system according to claim 1, wherein said data processor uses said rules for processing a plurality of visit records and corresponding associated first diagnostic codes using said rules to provide said plurality of visit records including second diagnostic codes compatible with said second assignment system by, identifying whether said first diagnostic codes are incompatible with said second assignment system and for visit records comprising incompatible codes, assigning second diagnostic codes to be compatible with said second assignment system and for visit records comprising compatible codes, using said first diagnostic codes as said second diagnostic codes.

11. A system for associating a diagnostic code to a record of a patient visit, comprising:
an interface processor for receiving a visit record comprising a first diagnostic code derived by using a first assignment system;
a source of sets of rules associated with particular time periods of validity, for processing said visit record to determine a second diagnostic code compatible with a second assignment system valid during a particular time period;
a data processor for processing said visit record and said first diagnostic code using said sets of rules to provide said visit record including said second diagnostic code, said second diagnostic code being valid for a particular time period encompassing a date of said visit; and
an output processor for initiating communication of data, representing said visit record and said second diagnostic code compatible with said second assignment system, to a destination system in response to a command.

12. A system according to claim 11, wherein an individual set of rules has a time period of validity determined by a start date and an end date.

13. A system according to claim 11, wherein said sets of rules associated with particular time periods of validity comprise a plurality of sets of rules for processing said visit record and said first diagnostic code, for processing said visit record to determine said second diagnostic code compatible with said second assignment system valid during the particular time period; and the data processor for processing said received record and said first diagnostic code using said sets of rules to provide said visit record including said second diagnostic code, said second diagnostic code being valid for a particular time period encompassing a date of said visit.

14. A system for associating a diagnostic code to a record of a patient visit, comprising:
an interface processor for receiving a visit record;
a source of sets of rules associated with particular time periods of validity, for processing said visit record to determine said diagnostic code compatible with an assignment system valid during a particular time period;
a data processor for processing said visit record using said sets of rules to provide said visit record including said diagnostic code, said diagnostic code being valid for a particular time period encompassing a date of said visit; and
an output processor for initiating communication of data, representing said visit record and said diagnostic code compatible with said assignment system, to a destination system in response to a command.

15. A system for associating a diagnostic code to a record of a patient visit, comprising:
an interface processor for receiving visit records individually including a first diagnostic code derived by using a first assignment system;
a source of rules for processing individual visit records to determine a second diagnostic code, for individual visit records, compatible with a second assignment system; and
a data processor for using said rules for processing said visit records and first diagnostic codes to provide visit records including second diagnostic codes compatible with said second assignment system by, grouping visit records into
retrieving sets of rules associated with particular time periods of validity, for processing said visit record to determine a second diagnostic code compatible with a second assignment system valid during a particular time period;
processing said visit record and said first diagnostic code using said sets of rules to provide said visit record including said second diagnostic code, said second diagnostic code being valid for a particular time period encompassing a date of said visit; and
initiating communication of data, representing said visit record and said second diagnostic code compatible with said second assignment system, to a destination system in response to a command.

20. A storage medium according to claim 19 containing computer readable instructions for performing said activities of the method of claim 19.

21. A method for associating a diagnostic code to a record of a patient visit, comprising the activities of:
receiving a visit record comprising a first diagnostic code, said first diagnostic code derivable from a first assignment system;
retrieving sets of rules associated with particular time periods of validity, for processing said visit record to determine a second diagnostic code, said second diagnostic code compatible with a second assignment system valid during a particular time period;
processing said visit record using said sets of rules to provide said visit record including said second diagnostic code, said second diagnostic code being valid for a particular time period encompassing a date of said visit; and
initiating communication of data, representing said visit record and said second diagnostic code compatible with said assignment system, to a destination system in response to a command.

22. The method of claim 21, further comprising:
obtaining a time dependent validity indicator relatable to the sets of rules, the time dependent validity indicator having a start date and an end date; and
testing said visit record comprising a first diagnostic code assignment date to verify that the first diagnostic code assignment date falls between the time dependent validity indicator start date and the time dependent validity indicator end date.

23. The method of claim 21, further comprising:
grouping said visit record into a cluster having common characteristics using characteristic information in said visit record; and
providing said second diagnostic code compatible with the second assignment system, corresponding to the visit record in the visit record cluster.

24. A machine-readable medium having stored thereon:
instructions adapted to process a visit record, the visit record comprising a first diagnostic code created by a first assignment system, using at least one of a set of rules to provide the visit record comprising a second diagnostic code; and
the set of rules adapted to process the visit record to determine the second diagnostic code compatible with a second assignment system comprising information adapted to derive a diagnostic code set.
